(19) United States
(12) Patent Application Publication
(10) Pub. No.: US 2023/0022980 A1
(43) Pub. Date: Jan. 26, 2023

(54) DOUBLE-DIGESTION ASSAY FOR ANALYZING LIGAND-DRUG CONJUGATES

(71) Applicant: Seagen Inc., Bothell (US)

(72) Inventors: Shawna Hengel, Bothell, WA (US); Michael Raymond Mylott, Jr., Bothell, WA (US); Michael Paul Waldron, Jr., Bothell, WA (US); James Aloysius Creegan, Bothell, WA (US)

(21) Appl. No.: 17/640,044

(22) PCT Filed: Sep. 2, 2020
(86) PCT No.: PCT/US20/49081

§ 371 Date: Mar. 3, 2022

Related U.S. Application Data
(60) Provisional application No. 62/895,599, filed on Sep. 4, 2019.

(51) Int. Cl. G01N 33/94; C07K 16/28; C12Q 1/37; C12Q 1/40; G01N 33/68

(52) U.S. Cl. CPC G01N 33/94; C07K 16/2803; C07K 16/2896; C12Q 1/37; C12Q 1/40; G01N 33/6851; G01N 2560/00

(57) ABSTRACT

Methods for analysis of a glucuronide ligand-drug conjugate are provided.

### CROSS REFERENCE TO RELATED APPLICATIONS

This application claims the benefit and priority to U.S. Provisional Patent Application No. 62/895,599, filed Sep. 4, 2019, the disclosure of which is hereby incorporated by reference in its entirety for all purposes.

### BACKGROUND

Ligand-drug conjugates (LDCs) are the focus of increasing interest for targeted therapy. LDCs are comprised of a cytotoxic agent, typically a small molecule drug with a high systemic toxicity, and a highly selective ligand for a tissue or cell-specific antigen (e.g., an antibody in the case of antibody-drug conjugates (ADCs)), linked together through a linker that is relatively stable in circulation, but releases the cytotoxic agent in the targeted environment. Antibody-drug conjugates (ADCs) hold great promise, especially in oncology, as the next generation of targeted therapies. Leveraging the immunologic specificity of antibodies to deliver highly potent cytotoxic agents to diseased tissue both improves antitumor activity and limits off target toxicities. This approach has now been used successfully in multiple FDA-approved ADCs, including gemtuzumab ozogamicin, inotuzumab ozogamicin, polatuzumab vedotin, brentuximab vedotin and ado-trastuzumab emtansine (Verma et al., 2012, Younes et al., 2010), and is the focus of numerous preclinical studies and clinical trials.

Development of improved LDCs typically requires multiple bioanalytical assays. Biotransformations, and drug or drug-linker stability, may be assayed by measuring the concentration of drug that is stably conjugated to the ligand over time, or after exposure to the biological environment using various analytic methods. Such assays require methods to release the drug or a portion thereof for subsequent measurement. This may be done by enzymatic cleavage. However, cleavage of some drugs and drug-linkers by a single enzyme can be inefficient, resulting in incomplete cleavage of the drug and drug linkers from LDCs.

Therefore, there is a need for improved means of cleaving drugs and drug-linkers from LDCs, that are suitable for use with appropriate analytic methods for detection and quantitation of released drugs or portions thereof.

### SUMMARY

In one aspect, provided herein are methods for releasing an analytic target from a ligand-drug conjugate (LDC) in a sample, comprising: providing a sample comprising an LDC, wherein said LDC comprises a ligand and an analytic target, wherein said analytic target comprises a drug molecule or a portion thereof, and wherein said analytic target is attached to said ligand by a linker comprising a glucuronide; contacting said sample with a proteolytic enzyme under conditions that promote exposure of said glucuronide; and contacting said sample with a glucuronidase, thereby inducing release of said analytic target from said LDC.

In some embodiments, the method further comprising the steps of: measuring an amount of said analytic target released from said LDC; and determining a concentration of said drug molecule or said portion thereof in said sample using said amount of said released analytic target.

In some embodiments, the step of measuring said amount of said analytic target released from said LDC comprises subjecting said analytic target to liquid chromatography-mass spectrometry (LC-MS). In some embodiments, the step of measuring said amount of said analytic target released from said LDC comprises subjecting said analytic target to liquid chromatography tandem mass spectrometry (LC-MS/MS). In some embodiments, said liquid chromatography is high performance liquid chromatography (HPLC).

In some embodiments, the method further comprises measuring said amount of said ligand in said sample; and determining said concentration of said drug molecule or said portion thereof in said sample by using said measured amount of said ligand.

In some embodiments, said sample is obtained from a cell or a mammalian subject. In some embodiments, the subject is a human subject.

In some embodiments, the sample is processed using a step selected from the group consisting of: affinity chromatography, size exclusion chromatography, ammonium sulfate precipitation, ion exchange chromatography, immobilized metal chelate chromatography, and immunoprecipitation. In some embodiments, said ligand is an antibody or a functional fragment thereof and said LDC or said internal standard are collected by affinity chromatography, size exclusion chromatography, ammonium sulfate precipitation, ion exchange chromatography, immobilized metal chelate chromatography, or immunoprecipitation.

In some embodiments, said ligand is an antibody or a functional fragment thereof and said LDC or said internal standard are collected by contacting said sample with an antigen, an antibody idiotype, an affinity magnetic bead, or a resin. In some embodiments, said resin is selected from a Protein A resin, a Protein G resin, and a Protein L resin.

In some embodiments, the method further comprises adding to said sample a fixed amount of an internal standard, where said internal standard comprises said ligand and a second analytic target, wherein said second analytic target is a labeled derivative of said LDC; contacting said sample with said proteolytic enzyme under conditions that promote exposure of said glucuronide; contacting said sample with said glucuronidase, thereby inducing release of said analytic target from said LDC and said second analytic target from said internal standard; measuring an amount of said second analytic target released from said internal standard; and measuring said amount of said analytic target released from said LDC based on said amount of said second analytic target released from said internal standard.

In some embodiments, said amount of said analytic target released from said LDC is determined by using said amount of said second analytic target released from said internal standard, wherein said amount of said analytic target released from said LDC correlates with a concentration of said drug molecule conjugated to an antibody in said LDC in said sample.

In some embodiments, the step of measuring said amount of said analytic target released from said LDC comprises using a standard curve generated from said LDC.

In some embodiments, said second analytic target has a different molecular weight than said analytic target. In some embodiments, said second analytic target has a different isobar than said analytic target. In some embodiments, said internal standard comprises a version of said LDC further comprising an isotopic label. In some embodiments, said isotopic label is stable or non-stable. In some embodiments, said isotopic label is deuterium or carbon 13.

In some embodiments, said glucuronidase comprises β-glucuronidase. In some embodiments, said first or second sample is contacted with β-glucuronidase at a concentration of at least 10,000 units/mL. In some embodiments, said first or second sample is contacted with β-glucuronidase at a concentration of no more than 100,000 units/mL. In some embodiments, said contacting said first or second sample with said glucuronidase occurs at a pH of about 5.

In some embodiments, said drug molecule is monomethyl auristatin E (MMAE).

In some embodiments, proteolytic enzyme is papain or trypsin. In some embodiments, said proteolytic enzyme is papain. In some embodiments, said proteolytic enzyme is trypsin.

In some embodiments, said contacting said first or second sample with said glucuronidase is at least six (6) hours at 37° C.

In a further aspect, provided herein are methods of determining stability of the ligand-drug conjugate (LDC), comprising: obtaining a first sample and a second sample from a single source at different time points; analyzing said LDC in said first sample and said second sample by the method of any of the embodiments, thereby determining the amount of said analytic target released from said LDC in said first sample and said second sample; and determining stability of said LDC by comparing the amount of said released analytic target in said first sample and said second sample.

In some embodiments, said comparing comprises determining a ratio of said amount of said released analytic target and said ligand in said first and said second samples.

In some embodiments, said first sample has a volume of at least 20 μL. In some embodiments, said sample, said first sample, or said second sample is a biological sample derived from mammalian tissue or aqueous mammalian fluid. In some embodiments, said biological sample is obtained from one of the following: plasma, serum, blood, tissue, tissue biopsy, feces, urine. In some embodiments, said biological sample is obtained from plasma. In some embodiments, said plasma was treated with said LDC. In some embodiments, said plasma is from a mammalian subject to whom said LDC has been administered. In some embodiments, the subject is a human subject. In some embodiments, the subject is a rat or cynomolgus monkey subject.

In some embodiments, said ligand is an antibody comprising an Fc region. In some embodiments, said antibody is an anti-CD48 antibody or an anti-CD228 antibody.

In a further aspect, provided herein are methods for quantifying a ligand-drug conjugate (LDC) in a sample, comprising: providing a sample comprising an LDC, wherein said LDC comprises a ligand and an analytic target, wherein said analytic target comprises a drug molecule or a portion thereof, wherein said analytic target is attached to said ligand by a linker comprising a glucuronide; adding to said sample an internal standard, wherein said internal standard is a labeled derivative of said LDC and comprises a second analytic target; contacting said LDC and said internal standard with a proteolytic enzyme under conditions that promote exposure of said glucuronide in said LDC; contacting said LDC and said internal standard with a glucuronidase, wherein said glucuronidase induces release of said analytic target from said LDC and said second analytic target from said internal standard; and determining an amount of said analytic target released from said LDC and an amount of said second analytic target released from said internal standard, wherein said amount of said analytic target released from said LDC correlates with said amount of said LDC in said sample.

In some embodiments, further comprising extracting said LDC and said internal standard from said sample prior to contacting said LDC and said internal standard with said proteolytic enzyme.

In some embodiments, the step of determining said amount of said analytic target released from said LDC and said amount of said second analytic target released from said internal standard comprises subjecting said analytic target and said second analytic target to liquid chromatography-mass spectrometry (LC-MS).

In some embodiments, the step of determining said amount of said analytic target released from said LDC and said amount of said second analytic target released from said internal standard comprises subjecting said analytic target and said second analytic target to liquid chromatography tandem mass spectrometry (LC-MS/MS).

In some embodiments, the liquid chromatography is high performance liquid chromatography (HPLC).

In some embodiments, the method further comprises measuring an amount of said ligand in said first or second sample; and determining a concentration of said drug molecule or said portion thereof in said first or second sample by using said measured amount of said ligand.

In some embodiments, the step of extracting said LDC and said internal standard is performed by at least one of: affinity chromatography, size exclusion chromatography, ammonium sulfate precipitation, ion exchange chromatography, immobilized metal chelate chromatography, and immunoprecipitation.

In some embodiments, said ligand is an antibody or a functional fragment thereof and said LDC or said internal standard are extracted by contacting said sample with a resin selected from a Protein A resin, a Protein G resin, and a Protein L resin.

In some embodiments, said amount of said analytic target released from said LDC is determined by using said amount of said second analytic target released from said internal standard, wherein said amount of said analytic target released from said LDC correlates with a concentration of said drug molecule conjugated to an antibody in said LDC in said sample.

In some embodiments, said amount of said analytic target released from said LDC is determined by using a standard curve generated from said LDC.

In some embodiments, said second analytic target has a different molecular weight than said analytic target.

In some embodiments, said internal standard comprises a version of said LDC further comprising an isotopic label. In some embodiments, said isotopic label is stable or non-stable.

In some embodiments, said isotopic label is deuterium or carbon 13.

In some embodiments, said glucuronidase comprises β-glucuronidase. In some embodiments, said sample is contacted with β-glucuronidase at a concentration of at least 10,000 units/mL. In some embodiments, said sample is contacted with β-glucuronidase at a concentration of no more than 100,000 units/mL. In some embodiments, said contacting said sample with said glucuronidase occurs at a pH of about 5.

In some embodiments, said drug molecule is monomethyl auristatin E (MMAE).

In some embodiments, said proteolytic enzyme is papain or trypsin. In some embodiments, said proteolytic enzyme is papain. In some embodiments, said proteolytic enzyme is trypsin. In some embodiments, said contacting said sample with said glucuronidase is at least six (6) hours at 37° C.

In some embodiments, said sample has a volume of at least 20 μL In some embodiments, said sample is a biological sample derived from mammalian tissue or aqueous mammalian fluid. In some embodiments, said biological sample is obtained from one of the following: plasma, serum, blood, tissue, tissue biopsy, feces, and urine. In some embodiments, said biological sample is obtained from plasma. In some embodiments, said plasma was treated with said LDC. In some embodiments, said plasma is from a human subject to whom said LDC has been administered.

In some embodiments, said ligand is an antibody comprising an Fc region. In some embodiments, said antibody is an anti-CD48 antibody or an anti-CD228 antibody.

### BRIEF DESCRIPTION OF THE DRAWINGS

FIG. 1A shows the percent drug recovery after ADC digestion by papain, trypsin, or pepsin followed by β-glucuronidase digestion. FIG. 1B shows the percent drug recovery at the LQC, MQC, and HQC levels after ADC digestion by papain followed by β-glucuronidase digestion.

FIG. 2A shows the concentration (ng/ml) of circulating CD228-targeting ADC in cynomolgus monkey plasma samples over 28 days. FIG. 2B shows a comparison of the concentration (ng/ml) of circulating CD228-targeting ADC plasma as determined by the double digest method and an ELISA assay.

FIG. 3 shows the concentration (ng/ml) of circulating CD48-targeting ADC in cynomolgus monkey plasma samples over 15 days.

FIG. 4 shows the concentration (ng/mL) of circulating CD48-targeting ADC in plasma samples collected from intravenously dosed human patients as determined by the double digest method and LC-MS/MS with reversed phase chromatography.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them below.

A “ligand-drug conjugate” or “LDC” refers to a ligand (e.g., an antibody) conjugated to a pharmaceutical agent, e.g., to a cytotoxic or cytostatic drug. “Ligands” include, but are not limited to, polymers, dendrimers, oligonucleotides, proteins, polypeptides, peptides, including cyclic peptides and glycopeptides, or any other cell binding molecule or substance. More specifically, ligands include aptamers (oligonucleotides or peptides), as well as various proteins, such as interferons, lymphokines, knottins, adnectins, anticalins, darpins, avimers, Kunitz domains, and centyrins. Additional ligands include hormones, growth factors, colony-stimulating factors, vitamins, and nutrient transport molecules. Suitable ligands include, for example, antibodies, e.g., full-length antibodies and antigen binding fragments thereof. Antibodies also include bispecific antibodies and multi specific antibodies.

An “antibody-drug conjugate” or “ADC” refers to an antibody, antigen-binding fragment, or engineered variant thereof conjugated to a pharmaceutical agent. Typically, antibody-drug conjugates bind to a target antigen (e.g., CD70) on a cell surface, followed by internalization of the antibody-drug conjugate into the cell and subsequent release of the drug into the cell. The antibody or antigen-binding fragment thereof may be covalently or non-covalently bound to the pharmaceutical agent. In specific embodiments, the drug in LDCs and particularly that in ADCs, is conjugated to the ligand, or more particularly the antibody, through a linker. The linker typically comprises residues resulting from conjugation to the drug and conjugation to the ligand separated by a chemical spacer. The chemical spacer may simply be a hydrocarbon chain, an alkenylene, (e.g., —(CH₂)n-, where n is a selected integer, or n is 2-10), or a heteroalkenylene chain containing one or more oxygens, carbonyls (C═O), sulfurs, or amino groups (e.g., NH or Nalkyl). The linker may be structurally more complex, for example, the linker may be substituted with a PEG (polyethylene glycol) group, or other hydrophilic group or may contain a cleavable group, e.g., a β-glucuronide that is cleavable by β-glucuronidase, such that cleaving the group, cleaves the linker.

The linker is a chemical species linking the ligand to the drug. Typically, the LDC is formed by two conjugation steps. A precursor to the linker, which is a heterobifunctional species, having two different reactive groups most often separated by a spacer and optionally substituted, is most often reacted with the drug molecule to form a linker-drug combination which retains one of the reactive groups. A heterobifunctional linker precursor contains the spacer between the two reactive groups with different reactivity. For example, a heterobifunctional linker precursor may contain an amine-reactive group at one end and a thiol reactive group at the other end. In another more specific example, a heterobifunctional linker precursor may contain a carbonate for reaction with an amine of the drug to form a carbamate. In other more specific examples, a heterobifunctional linker precursor may contain an azide or a N-hydroxysuccinimide ester (NHS ester or a sulfo-NHS ester) for reaction with an amine of the drug to form an amide. Each of such amine reactive groups can be paired in a linker precursor with a maleimide group, which under selected known conditions, is selective for reaction with thiols. After conjugation to the drug, one of the reactive groups remains in the linker-drug combination.

The linker-drug combination retaining the reactive group can then be used as a reagent for conjugation of the drug to the ligand. For example, a ligand conjugation reagent can contain a maleimide group for reaction with thiol groups on a ligand. More generally, the ligand conjugation reagent can contain any appropriate reactive groups for conjugation to groups on the ligand. The reactive groups may react, for example, with amine groups, with carboxylate groups, with thiol groups or with hydroxyl groups.

An “analytic target” refers to a drug or a portion thereof that is released or cleaved from a ligand-drug conjugate, and which is detected or measured (quantitated) by one or more known analytic techniques, e.g., mass spectrometry. The analytic target contains at least the drug or a portion thereof and may in addition contain a portion of the linker. The amount of analytic target is representative of the amount of the ligand-drug conjugate from which it is released or cleaved. More specifically the analytic target is the drug of the LDC or a portion of the drug of the LDC. In specific embodiments, where the drug is an auristatin, the analytic target can be a tetrapeptide released from the drug.

When an internal standard is used, an analytic target can be a drug or a portion thereof that is released or cleaved from the internal standard. Alternatively, an internal standard can be added as a pre-cleaved analytical target that does not require release or cleavage. In typical embodiments, an analytic target released from an internal standard can be differentiated from an analytic target released from a ligand-drug conjugate, for example, by having a different molecular weight and/or by being labeled.

The term “antibody” denotes immunoglobulin proteins that bind to an antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The terms “extract,” “extracted,” “extraction,” and “extracting” refer to isolation of an LDC or ADC from a heterogeneous sample comprising several proteins and other molecules. Any appropriate method or material known in the art that can selectively extract an LDC or ADC from a heterogeneous sample, particularly a biological sample, can be employed in the methods herein. Extraction, for example, can include: affinity chromatography, size exclusion chromatography, ammonium sulfate precipitation, ion exchange chromatography, immobilized metal chelate chromatography, and immunoprecipitation.

Binding of LDC or ADC to a resin that contains a species to which the ligand or antibody binds can be used for extraction. Antibody binding proteins can be used for extraction of ADCs. For example, extraction of an ADC from a sample may involve running the sample over a protein A column or contacting the sample with a protein A resin and thereafter removing the resin from the sample in order to capture the antibody, thereby extracting the ADC from the sample. With respect to ADC's, surface proteins protein A, protein G or protein L may be used for extraction. The structural requirements for binding of a given antibody to protein A, protein G or protein L are known in the art and one of ordinary skill in the art can select from among them, the appropriate surface protein for use with a given antibody. Materials useful in extractions using these proteins include resins, e.g., beaded agarose, or magnetic beads, or similar support material to which the protein A, protein G or protein L is covalently immobilized.

The terms “intracellularly cleaved” and “intracellular cleavage” refer to a metabolic process or reaction inside a cell on a ligand-drug conjugate (e.g., an antibody-drug conjugate), whereby the covalent attachment, e.g., the linker between the drug moiety and the ligand unit is broken, resulting in free drug, or other metabolite of the conjugate dissociated from the antibody inside the cell. The cleaved moieties of the drug-linker-ligand conjugate are thus intracellular metabolites.

The terms “release,” “released,” and “releasing” refer to extracellular cleavage of an analytic target from an LDC by the protease and glucuronidase cleavage method described herein. For a given LDC carrying (i.e. conjugated with) a given number of linker-drug combinations, the amount of analytic target released will typically vary with protease and/or glucuronidase concentration (see below) used in the release reaction, the temperature and pressure of the reaction (see below) and the reaction time employed. For consistency of results from sample to sample, the same protease and/or glucuronidase concentration and reaction conditions should be employed. Treatment with protease and/or glucuronidase as described herein need not release all analytic target from the LDC. All that is needed is to release an amount of analytic target that is sufficient for obtaining an accurate and precise measurement of the analytic target in view of the analytic method employed.

The terms “contact,” “contacted,” and “contacting” refer to adding acid or reagent to a sample, which may be a test sample or a control sample (including biological samples), so that the components of the sample are made available to the protease and/or glucuronidase reagent, and a reaction can thus occur.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. The term includes radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of Lu), chemotherapeutic agents, and toxins such as small molecule toxins or enzymatically active toxins of bacterial, fungal, plant or animal origin, including synthetic analogs and derivatives thereof. In certain embodiments, a cytotoxic agent is conjugated to an antibody or administered in combination with an antibody. Suitable cytotoxic agents are described further herein.

“Cytotoxic activity” refers to a cell-killing, a cytostatic or an anti-proliferative effect of a ligand-drug conjugate compound or an intracellular metabolite of a ligand-drug conjugate. Cytotoxic activity may be expressed as the IC₅₀ value, which is the concentration (molar or mass) per unit volume at which half the cells survive.

The term “patient” or “subject” includes human and other mammalian subjects such as non-human primates, rabbits, rats, mice, and the like and transgenic species thereof, that receive either prophylactic or therapeutic treatment.

The term “standard curve” or “calibration curve” refers to a graph used as a quantitative research technique. To generate the standard curve, multiple samples with known properties are measured and graphed, which then allows the same properties to be determined for unknown samples by interpolation on the graph. The samples with known properties are the standards, and the graph is the standard curve. Standard curves are of particular use when measuring the amount or concentration of an analyte in a sample that may contain an unknown amount of the analyte. The use of a standard curve alone represents the use of an external standard. As is understood in the art, the standard curve of a given analyte (i.e. the LDC) to be quantitated should generally span the concentration range of the analyte expected in the samples. Again as is understood in the art, samples used for preparing the standard curve are processed by the same steps as test samples and any control samples in which the analyte is to be measured. A standard curve can also be employed in combination with the use of an internal standard. In this case, a constant (or fixed) amount of the internal standard is added to each sample used to generate the standard curve of known analyte concentrations. The same constant amount of internal standard is added to each test sample and to any blanks or control samples. The details of use of standard curves (calibration curves) as an external standard and a combination of the use of a standard curve with addition of internal standard for quantitation of analytes by analytic methods, including MS, LC-MS and LC-MS/MS methods, is well known in the art. One of ordinary skill in the art understands how to use such analytic methods in the determination of concentrations of analytes in a variety of samples, including biological samples as discussed herein.

An “internal standard” is a chemical species that behaves in a selected assay similarly to the chemical species to be quantitated (i.e. LDC), but which is distinguishable from that chemical species in the analytic method being used. Typically, the internal standard is labeled to distinguish it from the chemical species to be quantitated, but the label employed does not significantly differentially affect its behavior compared to that of the chemical species to be quantitated. Preferably, anything that affects the measurement of the chemical species to be quantitated (e.g., analyte peak area) will also affect the measurement of the internal standard similarly. The ratio of the measurements of the chemical species to be quantitated and its internal standard preferably exhibits less variability than the measurement of the chemical species in a test sample. For use in mass spectrometry methods, the internal standard has a molecular weight that is different from the chemical species to be quantitated.

Most often labeling with stable isotopes, such as deuterium (²H) and carbon 13 (¹³C) is employed. Labeling must allow separate measurement of analyte and internal standard. Preferably, an isotopically labeled internal standard differs in molecular weight from the chemical species to be quantitated by at least 3 amu (i.e. labeling with 3 or more ²H or ¹³C). More specifically, labeling results in a difference in molecular weight of 6 amu or more. Internal standards can also be surrogates of the chemical species to be quantitated. Surrogate internal standards differ structurally from the chemical species to be quantitated by substitution of an atom or chemical group by a different group, for example the substitution of a methyl group or other small alkyl for a hydrogen, or the substitution of a halogen, e.g., a fluorine, for a hydrogen. Such surrogates may be of particular use where it is not possible to readily obtain an isotopically labeled internal standard.

The terms “determine,” “determined,” and “determining” refer to the ascertaining of the concentration or amount of a particular analyte based on a measurement of the amount of an analytic target and the known amounts of one or more correlative factors. As is understood in the art, an analyte concentration can be combined with the results of other measurements to determine other structural and physical properties of an analyte.

When trade names are used herein, the trade name includes the product formulation, the generic drug, and the active pharmaceutical ingredient(s) of the trade name product, unless otherwise indicated by context.

### Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### Assay for Analyzing a Ligand-Drug Conjugate (LDC)

Methods of quantifying the concentration of analytical targets (e.g., a drug) linked to a ligand are necessary for measuring the pharmacokinetics of LDCs. Such methods require the release of the analytical target from the ligand so that the target can be quantitated. A new linker technology that incorporates glucuronides has been developed. The glucuronide linker is cleaved by glucuronidase enzymes in vivo. However, the glucuronide linker is not as amenable to such methods in vitro, resulting in poor or incomplete release of the analytical targets and thus incorrect quantification. Complete and/or predictable target release is necessary to avoid quantitation bias of a specific target or drug conjugation site, resulting from inaccurate or under quantification of the target or drug at certain conjugation sites. This bias can result in inaccurate measurements of LDCs in biological samples. In order to avoid incomplete cleavage of the drug from the ligand, a multi-step approach can be used: i) the conjugated ligand (e.g., an antibody) is enzymatically digested to make the glucuronide in the linker more accessible using an enzyme; ii) the newly accessible glucuronide linker is treated with glucuronidase to release the drug; and iii) the newly released drug is subsequently quantified via mass spectrometry.

In one aspect, the present invention provides a method of analyzing a ligand-drug conjugate (LDC) in a sample, comprising the step of: a. providing a sample comprising an LDC, wherein said LDC comprises a ligand and an analytic target, wherein said analytic target comprises a drug molecule or a portion thereof, and wherein said analytic target is attached to said ligand by a linker comprising a glucuronide; b. contacting said sample with a proteolytic enzyme under conditions that promote exposure of said glucuronide; and c. contacting said sample with a glucuronidase, thereby inducing release of said analytic target from said LDC.

Ligand-Drug Conjugate (LDC) Sample Analysis

The present invention provides a method of analyzing a ligand-drug conjugate (LDC) in a sample. An LDC is a complex comprising a ligand and an analytic target. The analytic target comprises a drug molecule or a portion thereof. Various samples comprising an LDC or suspected to comprise an LDC can be subject to analysis using a method provided herein. In particular, a biological sample can be analyzed.

Sample

An LDC in a biological or non-biological sample can be analyzed by the methods provided herein. In preferred embodiments, the sample is a biological sample derived from a mammalian subject. In some embodiments, the sample is a biological sample derived from a human subject. In some embodiments, the biological sample is derived from mammalian tissue or aqueous mammalian fluid. Specifically, in some embodiments, the biological sample is obtained from one of the following: plasma, serum, blood, tissue, tissue biopsy, feces, and urine.

In some embodiments, the sample is a biological sample contacted with an LDC in vivo. For example, the sample can be a biological sample derived from a subject exposed to an LDC. In some embodiments, the sample is obtained at a specific time point after administration of an LDC. In some embodiments, the sample is obtained at multiple time points after administration of an LDC. In some embodiments, the sample is obtained before administration of an LDC.

In some embodiments, the sample is a biological sample contacted with an LDC in vitro. For example, the sample can be a cell or population of cells from a cell culture line. Any appropriate cell culture line, including primary or immortalized cell lines know in the art may be used, including but not limited to, T cells, JurKat cells, THP1 cells, HeLa, HEK293, A549, Vero, CHO, MDCK, or any combination thereof. In some embodiments, the in vitro sample is contacted with an LDC for a specific time period.

In some embodiments, the sample is a biological sample contacted with an LDC ex vivo. In some embodiments, the sample is contacted with an LDC for a specific time period. In some embodiments, a plurality of samples contacted with LDC for different periods are subject to analysis. In some embodiments, the sample is obtained before exposure to an LDC.

The sample can be any appropriate volume for the method as required. Sample volumes can be from about 1 μl to about 1 ml, 1-10 μl, 10-20 μl, 20-30 μl, 30-40 μl, 40-50 μl, 50-60 μl, 60-70 μl, 70-80 μl, 80-90 μl, 90-100 μl, 100-200 μl, 200-300 μl, 300-400 μl, 400-500 μl, 500-600 μl, 600-700 μl, 700-800 μl, 800-900 μl, or 900-1000 μl. Sample volumes can be at least 1 μl to at least 1 ml, 1-10 μl, 10-20 μl, 20-30 μl, 30-40 μl, 40-50 μl, 50-60 μl, 60-70 μl, 70-80 μl, 80-90 μl, 90-100 μl, 100-200 μl, 200-300 μl, 300-400 μl, 400-500 μl, 500-600 μl, 600-700 μl, 700-800 μl, 800-900 μl, or 900-1000 μl. In some embodiments, the sample is at least 1 μl, 5 μl, 10 μl, 20 μl, 30 μl, 40 μl, 50 μl, 60 μl, 70 μl, 80 μl, 90 μl, 100 μl, 200 μl, 300 μl, 400 μl, 500 μl, 600 μl, 700 μl, 800 μl, 900 μl, or 1000 μl. In some embodiments, the sample is at least 20 μl.

Proteolytic Enzymes

In some aspects, the LDC is contacted with a proteolytic enzyme to expose the glucuronide linker. For instance, if the LDC is an antibody drug conjugate (ADC), incubation of the ADC with pepsin results in cleavage of the antibody into a F(ab)₂ and non-linked Fc heavy chain fragments. Incubation of the ADC with papain results in cleavage of the antibody into two separate Fab fragments and an Fc fragment with the heavy chains still linked. The cleavage of the antibody into these fragments increases exposure of the glucuronide linker that links the drug to the antibody. Increased exposure of the glucuronide linker allows for increased cleavage of the linker with a glucuronidase enzyme, and increased drug release.

The proteolytic enzyme can be any appropriate protease, endopeptidase, exopeptidase, or dipeptidyl peptidase known in the art. Proteases are classified into seven general groups: serine proteases, cysteine proteases, threonine proteases, aspartic proteases, glutamic proteases, asparagine proteases, and metalloproteases, any of which can be used in the method disclosed herein. For instance, papain is a cysteine protease, while pepsin is an aspartic protease, both of which are suitable proteolytic enzymes for use in the method disclosed herein.

In some embodiments, the LDC is contacted with a proteolytic enzyme. In some embodiments, the proteolytic enzyme is pepsin, papain, trypsin, chymotrypsin, elastase, thermolysin, glutamyl endopeptidase, or neprilysin, or any combination thereof. In some embodiments, the proteolytic enzyme is papain. In some embodiments, the proteolytic enzyme is pepsin. In some embodiments, the proteolytic enzyme is trypsin.

β-Glucuronidase

In some aspects, the LDC comprises a glucuronide linker that links the drug to the ligand. Glucuronides, or glucuronosides, are molecules comprising a glucuronic acid linked to another molecule via a glycosidic bond. Glucuronidases are enzymes that cleave the glycosidic bond. In order to release the drug for quantification, the Exemplary glucuronidases for use in the present method include α-glucuronidase, β-glucuronidase, glycyrrhizinate beta-glucuronidase, glucuronosyl-disulfoglucosamine glucuronidase, or any combination thereof.

In some aspects, after an LDC comprising a glucuronide linker is contacted with a proteolytic enzyme, the LDC is then contacted with a glucuronidase to cleave the glycosidic bond. In some embodiments, the sample is contacted with β-glucuronidase.

One unit of β-glucuronidase from a mollusk source will liberate 1.0 μg of phenolphthalein from phenolphthalein glucuronide per hour at 37° C. at pH 5.0. One unit of β-glucuronidase from an E. coli source will liberate 1.0 μg of phenolphthalein from phenolphthalein glucuronide per hour at 37° C. at pH 6.8.

The concentration of glucuronidase used to contact the sample may be optimized. The concentration of glucuronidase can be at least 1,000 units/mL, 2,000 units/mL, 3,000 units/mL, 4,000 units/mL, 5,000 units/mL, 6,000 units/mL, 7,000 units/mL, 8,000 units/mL, 9,000 units/mL, 10,000 units/mL, 20,000 units/mL, 30,000 units/mL, 40,000 units/mL, 50,000 units/mL, 60,000 units/mL, 70,000 units/mL, 80,000 units/mL, 90,000 units/mL, or 100,000 units/mL, or more. The concentration of glucuronidase can be from 1,000-2,000 units/mL, 2,000-3,000 units/mL, 3,000-4,000 units/mL, 4,000-5,000 units/mL, 5,000-6,000 units/mL, 6,000-7,000 units/mL, 7,000-8,000 units/mL, 8,000-9,000 units/mL, 9,000-10,000 units/mL, 10,000-20,000 units/mL, 20,000-3000 units/mL, 30,000-40,000 units/mL, 40,000-50,000 units/mL, 50,000-60,000 units/mL, 60,000-70,000 units/mL, 70,000-80,000 units/mL, 80,000-90,000 units/mL, or 90,000 to 100,000 units/mL, or more. In some embodiments, the LDC is contacted with (3-glucuronidase at a concentration of at least 10,000 units/mL. In some embodiments, the LDC is contacted with β-glucuronidase at a concentration of at least 20,000 units/mL. In some embodiments, the LDC is contacted with β-glucuronidase at a concentration of at least 100,000 units/mL.

The LDC, or sample comprising the LDC, can be contacted or incubated with the proteolytic protease or glucuronidase at a pH from 2-3, 3-4, 4-5, 5-6, 6-7, 7-8, 8-9, or 9-10. The LDC can be contacted or incubated with the proteolytic protease or glucuronidase at a pH of about 2, 2.5, 3, 3.5, 4, 4.5, 5, 5.5, 6, 6.5, 7, 7.5, 8, 8.5, 9, 9.5, or 10. In some embodiments, the LDC is contacted with the glucuronidase at a pH of about 5.

The LDC, or sample comprising the LDC, can be contacted with the proteolytic enzyme or glucuronidase for at least 10 min, 15 min, 20 min, 25 min, 30 min, 45 min, 60 min, 75 min, 90 min, 105 min, 120 min or more. The LDC, or sample comprising the LDC, can be contacted with the proteolytic enzyme or glucuronidase for at least 2 hr, 2.5 hr, 3 hr, 3.5 hr, 4 hr, 4.5 hr, 5 hr, 5.5 hr, 6 hr, 6.5 hr, 7 hr, 7.5 hr, 8 hr, 10 hr, 12 hr, 14 hr, 16 hr, 18 hr, 20 hr, 22 hr, 24 hr, or more. In some embodiments, the LDC is contacted with the glucuronidase for at least 6 hrs.

The LDC, or sample comprising the LDC, can be contacted with the proteolytic enzyme or glucuronidase at about room temperature. The LDC, or sample comprising the LDC, can be contacted with the proteolytic enzyme or glucuronidase at about 20-40° C., 20-25° C., 25-30° C., 30-35° C., 35-37° C., 37° C., or 37-40° C. In some embodiments, the LDC is contacted with the glucuronidase at 37° C.

Ligand-Drug Conjugate (LDC)

Ligand

In some embodiments, the ligand is a protein having specific affinity to a target molecule. In some embodiments, the ligand is an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to target cells (e.g., cancer cell antigens, viral antigens, or microbial antigens) or other antibodies bound to tumor cells or matrix. In this regard, “functionally active” means that the fragment, derivative or analog is able to elicit anti-idiotype antibodies that recognize the same antigen as the antibody from which the fragment, derivative or analog is derived. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md.; Kabat E et al., 1980, J. Immunology 125(3):961-969).

Other useful antibodies include fragments of antibodies such as, but not limited to, F(ab′)₂ fragments, Fab fragments, Fvs, single chain antibodies, diabodies, tribodies, tetrabodies, scFv, scFv-Fv, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from murine monoclonal and human immunoglobulin constant regions. (See, e.g., U.S. Pat. Nos. 4,816,567; and 4,816,397, each of which is incorporated herein by reference in its entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060; each of which is incorporated herein by reference in its entirety.

Completely human antibodies are particularly desirable and can be produced using transgenic mice that are incapable of expressing endogenous immunoglobulin heavy and light chains genes, but which can express human heavy and light chain genes.

Antibodies include analogs and derivatives that are either modified, i.e. by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

Antibodies immunospecific for a cancer cell antigen can be obtained commercially or produced by any method known to one of skill in the art such as, e.g., chemical synthesis or recombinant expression techniques. The nucleotide sequences encoding antibodies immunospecific for a cancer cell antigen can be obtained, e.g., from the GenBank database or a database like it, the literature publications, or by routine cloning and sequencing.

In certain embodiments, useful antibodies can bind to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD2O, CD22, CD28, CD30, CD70, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

In some embodiments, the ligand is a receptor ligand. The receptor ligand can have a binding partner that is enriched in a specific cell type, tissue or organ. The ligand can be a naturally occurring agonist or antagonist of a receptor, or a synthetic molecule that has an affinity to the receptor. The receptor ligand can be a protein, nucleic acid or other receptor ligand such as a peptide, vitamin, and carbohydrate. In one embodiment, the ligand is folate that has affinity to a folate receptor.

In some embodiments, the ligand is a targeting moiety that has been used and developed for targeting a drug to a target organ or tissue. Such site-specific ligands known in the art can be used and adopted in the method provided herein.

In some embodiments, the ligand is an antibody comprising an Fc region. In some embodiments, the antibody is an anti-CD48 antibody or an anti-CD228 antibody.

Drug

The drug of the LDC can be any cytotoxic, cytostatic or immunosuppressive drug also referred to herein as a cytotoxic, cytostatic or immunosuppressive agent. The drug has a functional group, such as an amino, alkyl amino group or carboxylate that can form a bond with an appropriate reactive group of a reagent precursor containing the linker, such as an amine group, a carboxylic acid group, a sulfhydryl group, a hydroxyl group or an aldehyde or ketone group. In an embodiment, the drug is conjugated to a linker to generate an amide or a carbamate. In an embodiment, the drug is conjugated to a linker by an amide bond. In an embodiment, the drug contains a single amide bond. In an embodiment, the drug is conjugated to the linker by a carbamate and the drug contains an amide bond.

Useful classes of cytotoxic or immunosuppressive agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like. Particularly useful classes of cytotoxic agents include, for example, DNA minor groove binders, DNA alkylating agents, and tubulin inhibitors. Exemplary cytotoxic agents include, for example, auristatins, camptothecins, duocarmycins, etoposides, maytansines and maytansinoids (e.g., DM1 and DM4), taxanes, benzodiazepines (e.g., pyrrolo[1,4]benzodiazepines (PBDs), indolinobenzodiazepines, and oxazolidinobenzodiazepines) and vinca alkaloids. Select benzodiazepine containing drugs are described in WO 2010/091150, WO 2012/112708, WO 2007/085930, and WO 2011/023883.

In an exemplary embodiment, the drug is a peptidic drug containing one or more, two or more, three or more or four or more amino acid groups. In an exemplary embodiment, the drug is a peptidic drug containing an N-terminal, N-methylated amino acid group. In a further exemplary embodiment, the drug is a peptidic drug having an N-terminal, N-methylated amino acid with an alkyl side group. In a further exemplary embodiment, the drug is a peptidic drug having an N-terminal, N-methylated alanaine, N-methylated isoleucine, N-methylated leucine or N-methylated valine. In a further exemplary embodiment, the drug is a peptidic drug having an N-terminal, N-methylated valine.

In some embodiments, the drug is an auristatin. Auristatins include, but are not limited to, AE, AFP, AEB, AEVB, MMAF, and MMAE. The synthesis and structure of auristatins are described in U.S. Patent Application Publication Nos. 2003-0083263, 2005-0238649 2005-0009751, 2009-0111756, and 2011-0020343; International Patent Publication No. WO 04/010957, International Patent Publication No. WO 02/088172, and U.S. Pat. Nos. 7,659,241 and 8,343,928; each of which is incorporated by reference herein in its entirety and for all purposes. Exemplary auristatins of the present invention bind tubulin and exert a cytotoxic or cytostatic effect on the desired cell line. In an embodiment, exemplary auristatins contain an N-terminal, N-methylated amino acid. More specifically, exemplary auristatins contain an N-terminal N,N-methylated amino acid with an alkyl side chain, such as alanine, isoleucine, leucine, or valine. Yet more specifically, exemplary auristatins contain an N-terminal, N-methylated valine.

In some embodiments, the antibody-drug conjugate can be a pegylated monomethyl auristatin E (DPR-PEG-gluc-carbamate-MMAE)-conjugated antibody. The MMAE linker is pegylated and contains diaminoproprionic acid and β-glucuronide which is cleavable by β-glucuronidase, see structure below.

The mal-peg-carbamate-MMAE has the structure:

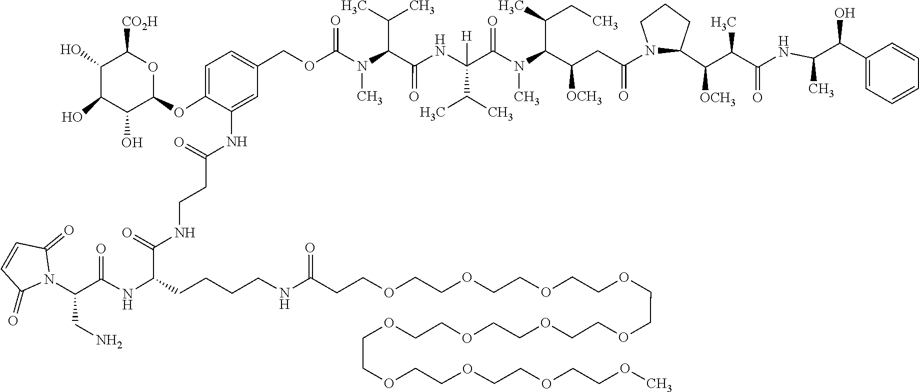 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)[C@H](CCCCNC(=O)CCOCCOCCOCCOCCOCCOCCOCCOCCOCCOCCOCCOC)NC(=O)[C@H](CN)N2C(=O)C=CC2=O)c1)C(C)C]

Other individual cytotoxic or immunosuppressive agents include, for example, an androgen, anthramycin (AMC), asparaginase, 5-azacytidine, azathioprine, bleomycin, busulfan, buthionine sulfoximine, calicheamicin, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16 and VM-26.

Suitable cytotoxic agents also include DNA minor groove binders (e.g., enediynes and lexitropsins, a CBI compound; see also U.S. Pat. No. 6,130,237), duocarmycins (see U.S. Publication No. 20060024317), taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

Examples of anti-tubulin agents include, but are not limited to, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. Maytansine and maytansinoid are another group of anti-tubulin agents. (ImmunoGen, Inc.; see also Chari et al., 1992, Cancer Res. 52:127-131 and U.S. Pat. No. 8,163,888).

Exemplary auristatin drugs have the following formula or a pharmaceutically acceptable salt thereof wherein the wavy line indicates site of attachment to the linker:

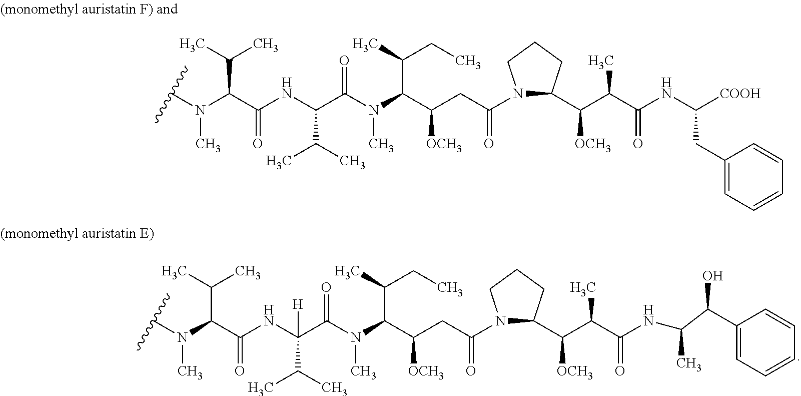 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C ; *N(C)[C@H](C(=O)N[C@@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Alternative auristatin drugs for conjugation to a ligand through a linker have the following formula or a pharmaceutically acceptable salt thereof, where the wavy line indicates the site of attachment to the linker:

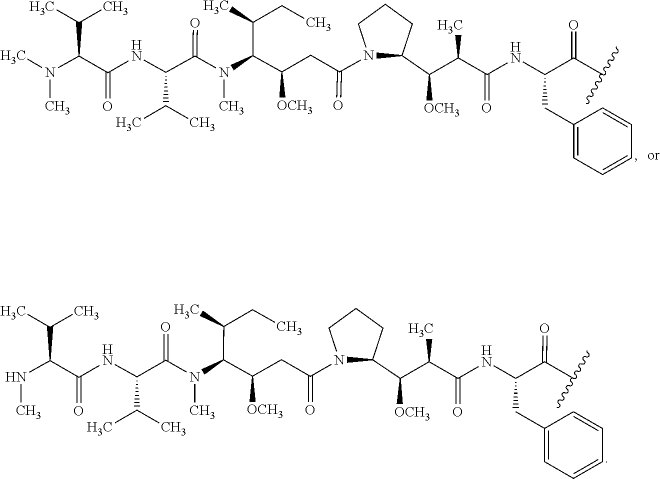 [SMILES: *C(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C ; *C(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@@H](NC)C(C)C)C(C)C]

Additional cytotoxic compounds useful for the preparation of LDCs and particularly useful for the preparation of ADCs are those described in U.S. Pat. No. 6,884,869, which is incorporated by reference herein in its entirety, particularly for descriptions of cytotoxic compounds. Additional description therein describes preparation of drug conjugates with the cyctotoxic compounds described.

Linker

General procedures for linking a drug to linkers are known in the art. See, for example, U.S. Pat. Nos. 8,163,888, 7,659,241, 7,498,298, U.S. Publication No. US20110256157 and International Application Nos. WO2011023883, and WO2005112919.

The linker can be cleavable under intracellular conditions, such that cleavage of the linker releases the therapeutic agent from the ligand in the intracellular environment (e.g., within a lysosome or endosome or caveolea). The linker can be, e.g., a peptidyl linker that is cleaved by an intracellular peptidase or protease enzyme, including a lysosomal or endosomal protease. Intracellular cleaving agents can include cathepsins B and D and plasmin (see, e.g., Dubowchik and Walker, Pharm. Therapeutics 83:67-123, 1999). For example, a peptidyl linker that is cleavable by the thiol-dependent protease cathepsin-B, which is highly expressed in cancerous tissue, can be used (e.g., a linker comprising a Phe-Leu or a Val-Cit peptide). The linker can also be a carbohydrate linker, including a sugar linker that is cleaved by an intracellular glycosidase (e.g., a glucuronide linker with a glucuronic acid which is cleavable by a glucuronidase). Ligand-drug conjugates with (3-glucuronide linkers are generally described in U.S. Pat. No. 8,039,273, hereby incorporated by reference in its entirety.

A glucuronic acid is generally represented as:

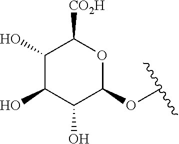 [SMILES: *O[C@@H]1O[C@H](C(=O)O)[C@@H](O)[C@H](O)[C@H]1O]

An antibody can be conjugated to one or more linker via any appropriate reactive group, e.g., via an amine group (for example, an N-terminal amino group or an amine group of an amino acid side group, such as lysine), a thiol group (—SH, for example, that of a cysteine residue), a carboxylate (for example, a C-terminal carboxylate, or that of an amino acid side chain, such as glutamic acid) or a hydroxyl group (for example of a serine residue), of the antibody.

In exemplary ADCs, monomethyl auristatin E is conjugated through a protease cleavable peptide linker to an antibody. The linker may, in addition, contain chemical groups that modulate solubility or pharmacokinetics. For example, an exemplary linker is pegylated. An exemplary linker-drug combinations is:

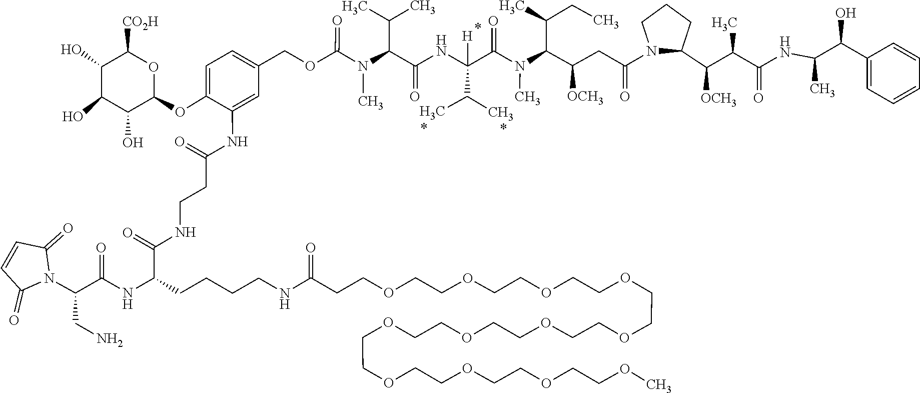 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)[C@H](CCCCNC(=O)CCOCCOCCOCCOCCOCCOCCOCCOCCOCCOCCOCCOC)NC(=O)[C@H](CN)N2C(=O)C=CC2=O)c1)C(C)C]

DPR-PEG-gluc-carbamate-MMAE, wherein the linker is pegylated and contains a glucuronic acid (cleavable by glucuronidase) and wherein the maleimide group of the linker can react with thiol groups of a ligand. In LDCs containing the glucuronic acid linker-drug combinations, treatment with glucuronidase as described herein releases the entire drug MMAE from DPR-PEG-gluc-carbamate-MMAE. Internal standards for LDCs and ADCs can be prepared by labeling of such linker-drug combinations, wherein the label is released on treatment with β-glucuronidase as described herein. Exemplary internal standards for LDC and ADC conjugated to mc-MMAE, include those that are deuterated or labeled with ¹³C in the MMAE released. In the above structures, sites for possible ¹³C labeling or deuterium labeling are shown by “*.”

Quantitation methods herein generally employ the release of a fragment of a LDC, designated as an analytic target herein, which represents the entire LDC, and which analytic target is quantitated. Quantitation of the analytic target allows one to measure the amount of analytic target released, the amount of analytic target in the LDC in a sample and/or the amount of LDC in a sample. In some determinations, it is necessary to know or to determine, by appropriate known methods, the amount of ligand in a sample or to know or to determine, by appropriate methods, the number (or average number) of drug molecules conjugated to a given LDC. More specifically, the analytic target herein is the drug molecule of the LDC or a portion of the drug molecule of the LDC. Drugs are conjugated to the ligand in an LDC by a linker species, so an analytic target may also include a portion of or the entire linker in addition to the drug or portion thereof. In specific embodiments, herein the analytic target is the drug conjugated to the LDC. In specific embodiments, herein the analytic target is a portion of the drug conjugated to the LDC. In specific embodiments herein, the drug is a peptide or derivative thereof and the analytic target is the peptide drug or a peptide portion of the peptide drug. In specific embodiments, where the drug is a peptide or derivative thereof, the analytic target is a dipeptide or derivative thereof, a tripeptide or derivative thereof, or a tetrapeptide or derivative thereof.

Measurement of Analytic Target

In some embodiments, the method involves the step of measuring an analytic target in a sample. An analytic method appropriate for quantitation of the analytic target in the concentration range that is expected to be encountered in samples can be used.

In some embodiments, an LDC or an internal standard is extracted from the sample prior to the measurement of the analytic target. The analytic target can be collected by affinity chromatography, size exclusion chromatography, ammonium sulfate precipitation, ion exchange chromatography, immobilized metal chelate chromatography, or immunoprecipitation. In some embodiments, an LDC or an internal standard includes an antibody or a functional fragment as a ligand. In those cases, the LDC or the internal standard can be collected by contacting the sample with an antigen an antibody idiotype or a resin selected from a Protein A resin, a Protein G resin and a Protein L resin.

In some embodiments, the analytic target is detected and quantified using liquid chromatography/mass spectrometry (LC/MS) or liquid chromatography tandem mass spectrometry (LC-MS/MS) methods. More specifically, tandem mass spectrometry (MS/MS) methods are employed. In MS/MS methods, one or more fragment ions of a selected parent ion of the analytic target are monitored. A parent ion of the analytic target is selected as known in the art in a first MS step and that parent ion is subjected to fragmentation, typically collision-induced fragmentation, to generate one or more fragment ions each of which can be quantitated by measurement, for example, of the ion current associated with each fragment to generate ion current peaks as a function of mass (m/z). Integrated peak areas of a fragment can be measured for quantitation of the chemical species from which the parent ion and one or more fragment ions thereof derive. In application to measurement of analytic target herein, the one or more fragments derive from the parent ion of the released analytic target.

Any MS/MS method can be employed for quantitation of analytic targets herein, but methods employing a triple quadrupole or a quadrupole-ion trap are more typically employed. Mass spectrometers used in the methods herein can be operated to monitor the entire mass spectrum of a sample, or more typically a selected portion thereof of interest. Particularly in MS/MS methods, the signal (e.g., ion current) from one or more fragment ions of a selected parent ion may be monitored. Selected reaction monitoring (SRM) operation can be used in which a single fragment ion generated from a selected parent ion is monitored. Alternatively, multiple reaction monitoring (MRM) operation can be used in which more than one fragment ion generated from a selected parent ion is monitored. The use of the term fragment ion relates to ions generated in MS/MS by the dissociation or fragmentation of a selected ion. It will be appreciated that methods are known in the art and used for quantitation of analytes that involve reacting selected parent ions to more generally generate product ions which include fragment ions as well as other product ions that are not fragment ions. MS/MS methods which generate all such product ions can be analogously employed in the methods herein.

In some embodiments, a liquid chromatography method appropriate for use in quantitation of analytic targets in various samples is used.

In some embodiments, the method involves use of standard curves (calibration curves) as an external standard and a combination of the use of a standard curve with addition of internal standard for quantitation of analytes by MS, LC-MS and LC-MS/MS methods. In some embodiments, the standard curves can be used to determine concentrations of analytes in a variety of samples, including biological samples as discussed herein. Specifically, the amounts of analytes from internal standard can be used to determine the amounts of analytes from an LDC. In particular embodiments, the amounts of analytes from internal standard are used to generate standard curves for use in determination of amounts of analytes from an LDC. In these embodiments, analytes from internal standard and analytes from an LDC can be differentiated by labeling.

Concentration Assay

In some embodiments, the method further comprises the step of determining the concentration of an LDC in a sample. The present invention also provides a method for determining, in a sample, the concentration of a drug that is conjugated to a ligand in an LDC.

The quantitation analysis preferably includes calibration within the assay. A standard curve can be generated, for example, by preparing a series of at least 6 samples with increasing concentrations of LDC. The internal standard is added to the standard curve samples, which are then processed by the protein A and LC-MS/MS methods described above. The peak area for each standard is divided by the peak area obtained for the internal standard, and the resultant peak area ratios are plotted as a function of standard concentrations. In some embodiments, at least 6 data points are fitted to a curve using, for example, linear regression analysis.

Stability Assay

In some embodiments, the method is used to determine stability of an LDC.

In an exemplary assay, the LDC is placed in sterile plasma and incubated at 37° C. At the beginning of the incubation and at varying timepoints from 1 hour to 1 week or longer, an aliquot is removed and at frozen at −80° C. Upon completion of the timepoints, the samples are subjected to a protein purification method that will specifically extract the ligand and conjugated drug. For example, an antibody-drug conjugate may be passed over a protein A affinity resin to capture the antibody, and subsequently the resin is washed with buffer. After capture of the ligand-drug conjugate, the drug is released from the captured ligand by treatment with a protease and β-glucuronidase. The released drug can then be quantified by standard LC-MS methodology, and the quantity of drug measured at each timepoint divided by the quantity of drug measured for the pre-incubation aliquot can be used to determine the percentage of drug remaining conjugated to the ligand at each timepoint. The precision of this assay can be improved by including an internal standard ligand-drug conjugate which is prepared using an isotopically labeled version of the same drug-linker, such that the drug which is released from it can be detected independently in the LC-MS assay from the drug released from the test drug-linker by virtue of its mass difference. This isotopically labeled internal standard ligand-drug conjugate is added to each sample in equal amounts immediately prior to the ligand capture step (e.g., protein A). The quantitation of the drug or a portion of the drug released from the test LDC is then performed using the internal standard by conventional liquid chromatography-mass spectrometry (LC-MS/MS) techniques. Mass spectrometry techniques for use in pharmacokinetics assays are known in the art. (See, for example, Want et al., Spectroscopy 17:681-691 (2003); Okeley et al., Clin Cancer Res. 16: 888-897 (2010); Singh et al., DMD (2017); Alley et al., Bioconjugate Chem. 19:759-765 (2008).).

In other embodiments, an LDC is administered to a subject and samples are obtained from the subject at different time points after administration of the LDC. The plurality of samples are subject to the methods provided herein for measurement of analytic target from the LDC. In some embodiments, internal standard is administered together with the LDC. Amounts of LDC in the samples can be compared and used to determine stability of the LDC over time.

In some embodiments, an LDC is added to a sample ex vivo. Samples are collected after various time points after addition of the LDC. The plurality of samples are subject to the methods provided herein for measurement of analytic target from the LDC. In some embodiments, internal standard is added to the sample together with the LDC. Amounts of LDC in the samples can be compared and used to determine stability of the LDC ex vivo over time.

Other Assays

The method provided herein can be used to determine average number of drugs per ligand. For example, average number of drugs per ligand can be measured by dividing the concentration of ligand conjugated drug, obtained by the methods described herein, by the concentration of ligand.

In other embodiments, the cleavage methods and related analytic methods described herein can be used in a variety of experiments that rely on the determination of the amount of LDC in a sample or determining the amount of drug conjugated to an LDC. The methods herein can, for example, be used for determining release kinetics of drugs from LDCs in the context of developing clinical agents for treatments of diseases or disorders. The methods herein can also be used for studying the pharmokinetics of an LDC. The methods herein can be used to assess the use of LDCs in clinical applications.

### Kits

In another aspect, a kit for measurement of LDC in a sample or for measurement of the amount of drug conjugated to an LDC is provided. A kit comprises one or more chemical and typically more than one chemical component useful for carrying out an assay as described herein. In a kit, the different chemical components are typically provided in selected amounts in separate containers packaged together and optionally including instructions for carrying out the assay. The amounts of chemical components in a given kit are typically provided in selected amounts to carry out a selected number of assays for each kit. For example, each kit can be designed to carry out one assay and thus is provided with a sufficient amount of the chemical species to carry out all steps in a given assay. Kits are optionally also provided with reagents or solvents needed for carrying out an assay. Kits can be provided, for example, with reagents for extracting a given LDC or a class of LDC from samples. In an embodiment, kits herein comprise an appropriately labeled internal standard for any given LDC, including any ADC. The internal standard of the kit can be an isotopically labeled LDC, where the label is positioned in the drug. Such kits may also contains unlabeled LDC for preparation of standard curves.

In another embodiment, kits comprise a reagent comprising a labeled linker-drug combination containing a reactive group for conjugating the linker and drug to any selected ligand, including any selected antibody. More specifically, the reagent is labeled in the drug or a portion thereof so that on release of analytic target the label is released with the analytic target. The kit optionally further contains reagents or solvent for carrying out a conjugation with a selected ligand or antibody. A kit may also contain unlabeled linker-drug reagent for preparation of unlabeled LDC. The kit may further contain unlabeled or labeled analytic target, e.g., the drug or the portion of the drug released by protease and glucuronidase treatment. In a specific embodiment, a kit contains an an isotopically labeled DPR-PEG-gluc-carbamate-MMAE for conjugation to any selected ligand or antibody to serve as an internal standard for measurement of L-DPR-PEG-gluc-carbamate-MMAE. Such kits can be used as research aids for development of LDCs suitable for clinical use. Such kits can also be employed in clinical application where there is a need to monitor LDC or LDC drug loading in a patient.

In some embodiments, a kit may comprise a pair of reagents for conjugating the linker-drug combination, and the ligand, in separate packaging, as well as the reagents necessary for a single conjugation reaction. The kit may optionally include solvent or buffer for carrying out reactions and instructions for use. Methods for conjugation of ligands and drug-linkers are known in the art. (See, for example, Lyon et al., Methods in Enzymology, vol. 52, pgs. 123-138, 2012; Sun et al., Bioconjugate Chem. 16:1282-1290, 2005.) Internal standards and reagents can be isotopically labeled with either stable or unstable isotopes. Stable isotopes include, but are not limited to, ²H, ¹³C, and ¹⁵N. Radioactive or unstable isotopes include, but are not limited to, ³H, ¹⁴C, and ¹²N.

Alternatively, an internal standard may be distinguished from the LDC by a structural modification that confers a different molecular weight, but is not isotopically labeled. For example, an internal standard may comprise a methyl group or a halogen instead of hydrogen at a position in the analytic target. As is appreciated in the art any internal standard for a given analyte used must be assessed to ensure that it behaves as the analyte in a given analytic method.

### EXAMPLES

The following examples are provided by way of illustration not limitation.

### Example 1

### Double Digestion Assay

An improved method of releasing glucuronide-linked drugs from ADCs was developed. Drug release assays using only β-glucuronidase digestion have inefficient drug release from the antibody and thus poor drug recovery. Therefore, a double digestion of ADCs, using an first enzyme digest prior to the β-glucuronidase digestion step, was developed to increase the overall drug release and recovery.

### Materials and Methods

ADCs in TBS were treated with Rapigest™ surfactant (Waters, Milford Mass.) and dithiothreitol (DTT) for 1 hour at 60° C. 0.1 M idodoacetic acid (IAA) in 0.1 NaOH was added to adjust the solution to pH 5 and samples were incubated at room temperature for 30 min. The ADCs were digested using 2.5 μg trypsin at 37° C. for 90 min. The digestion was stopped by adding 2N HCl. Next, 100 units of β-glucuronidase were added and the samples incubated at 37° C. The samples were evaporated under N₂ at 60° C. Cold 1% ammonia in methanol was added and the samples incubated at room temperature for 20 min before being centrifuged at 5,000 rpm for 5 min. The supernatant was transferred to a low binding plate and evaporated again under N₂ at 35° C. Samples were reconstituted with 20:80:0.1 acetonitrile/water/formic acid and centrifuged at 5,700 rpm for 5 min at 4° C. The amount of recovered drug was quantified using LC-MS/MS with reversed phase chromatography, H2O+0.1% Formic acid and acetonitrile+0.1% Formic acid mobile phases, and a Sciex 6500 triple quadrupole mass spectrometer (SCIEX).

### Results

Trypsin protease treatment prior to the β-glucuronidase treatment step resulted in only a 25% recovery of the drug. Further optimization of the pH during the β-glucuronidase step resulted in approximately 30-40% recovery of the drug.

### Papain Protease Cleavage

To increase the amount of recovered ADC, the protease used prior to the β-glucuronidase treatment step was altered. Samples with ADCs were treated with 400 μg of papain (Worthington NO. LS003119, >15 units/mg protein) or pepsin using the method described above and compared to samples treated with trypsin. Samples were treated with papain for 0.5, 2, or 4 hr. Samples treated with pepsin for 0.5 hr were incubated at 60° C. for 0.5 hr. Samples treated with pepsin for 2 hr were incubated at 60° C. for 1 hr followed by 37° C. for 1 hr. Samples treated with pepsin for 4 hr were incubated at 60° C. for 2 hr followed by 37° C. for 2 hr. Samples treated with trypsin for 1.5 or 3.5 hr were incubated at 37° C. In addition, the incubation time of the proteases was optimized and some samples were treated with or without Rapigest or DTT and IAA. The incubation conditions and drug recovery results are shown in Table 1.

TABLE 1

| | Protease | Protease | | DTT | Drug |
|---|---|---|---|---|---|
| Protease | Time (Hr) | Temp | Rapigest | IAA | Recovery |
| Papain | 0.5 | 60 | No | No | 77.4% |
| Papain | 2 | 60-37 | No | No | 82.3% |
| Papain | 4 | 60-37 | No | No | 78.4% |
| Papain | 0.5 | 60 | No | No | 76.2% |
| Papain | 2 | 60-37 | Yes | No | 80.7% |
| Papain | 4 | 60-37 | Yes | No | 75.5% |
| Trypsin | 1.5 | 37 | Yes | Yes | 36.6% |
| Trypsin | 3.5 | 37 | Yes | Yes | 37.2% |
| Trypsin | 1.5 | 37 | Yes | No | 43.4% |
| Trypsin | 3.5 | 37 | Yes | No | 39.3% |

The use of papain as the protease increased drug recovery from 36-43% to 75-82%. Thus, papain was likely more efficient at exposing the glucuronide linker to the β-glucuronidase for linker cleavage than trypsin was.

### Papain vs Pepsin Cleavage

Next, papain and pepsin were compared to each other to determine which was more efficient. Samples with ADCs were treated with papain or pepsin using the method described above.

The samples with pepsin were treated at 37° C. for 1.5 hr, 2.5 hr, 3.5 hr, 4.5 hr, or 6 hrs and followed with β-glucuronidase treatment overnight (16+ hr). One sample was treated with pepsin for 3.5 hr at 37° C. followed by papain for 2.5 hr at 37° C. and then β-glucuronidase overnight.

The samples with papain were treated at 60° C. for 1 hr, or 60° C. for 1 hr followed by 37° C. The papain samples were treated with β-glucuronidase for 2 hr, 3 hr, 6 hr, or overnight (16+ hr).

The incubation conditions and drug recovery results are shown in Table 2. A dash (-) indicates no treatment under those conditions.

TABLE 2

| Pepsin | Papain | Glucuronidase | Drug |
|---|---|---|---|
| Time hr (37° C.) | Time hr (60° C.) | Time hr (37° C.) | Recovery |
| 1.5 | — | 16+ | 23.3% |
| 2.5 | — | 16+ | 4.6% |
| 3.5 | — | 16+ | 22.0% |
| 4.5 | — | 16+ | 26.1% |
| 6 | — | 16+ | 20.3% |
| 6 | — | 16+ | 28.4% |
| 3.5 | 2.5 (37° C.) | 16+ | 36.4% |
| — | 1 | 2 | 23.5% |
| — | 1 | 4 | 35.3% |
| — | 1 | 6 | 66.0% |
| — | 1 | 16+ | 78.2% |
| — | 1 (60° C.) + | 16+ | 78.6% |
| | 1 (37° C.) | | |

The pepsin alone treatment combined with an overnight β-glucuronidase incubation step resulted in 4-28% drug recovery. The papain alone treatment resulted in 23-78% drug recovery, with increased drug recovery in the samples with longer β-glucuronidase incubation, e.g., the papain sample treated with β-glucuronidase for 2 hrs had a 23.5% drug recovery, while the papain sample treated with β-glucuronidase for 16+ hrs had a 78.2% drug recover. Increasing the papain incubation time by an additional hour at 37° C. did not result in a significant increase in drug recovery.

Pepsin did not help improve recovery even in combination with papain. The minimum amount of time for best β-glucuronidase hydrolysis was greater than 6 hr, and best recovery was observed in samples incubated overnight (16+ hrs). Thus, papain treatment alone resulted in the best drug recovery.

### Example 2

### Recovery of Drug Target from Optimized Double Digestion Conditions

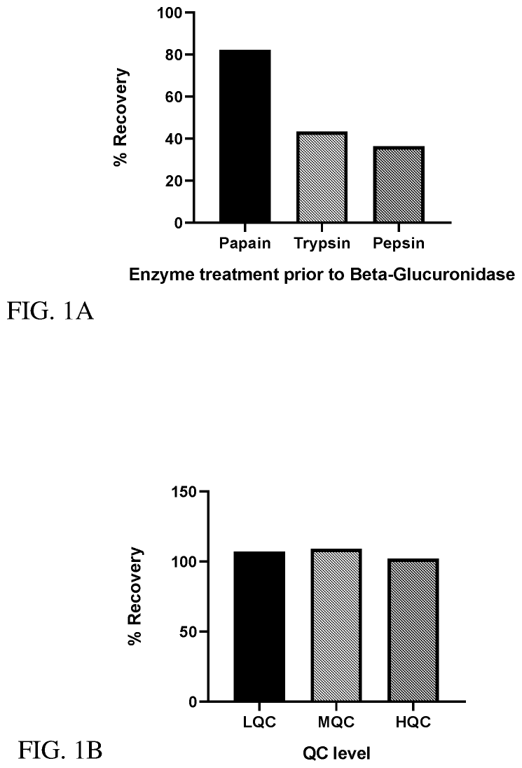

Next, recovery of the drug after the double digestion was further optimized. ADCs were incubated with papain, trypsin, or pepsin as previously described, followed with release of the drug using β-glucuronidase to screen for improved recovery of the drug. Recovery of the drug was quantified via LC-MS/MS as previously described. Percent recovery of the drug was determined by analysis of released drug as compared to LC-MS/MS analysis of an equivalent column loading of the drug, assuming 100% release of the drug from the ADC (FIG. 1A). Similar to Example 1, digestion with papain resulted in the greatest drug recovery, about 80%, while trypsin resulted in only about 40% drug recovery, and pepsin resulted in about 35% drug recovery.

Based on the normalized drug recovery results, papain was selected as the enzyme to be used in the first digestion step. Next, conditions of the assay were further optimized. ADCs were added to plasma and digested with papain and β-glucuronidase. Recovery of the drug was quantified via LC-MS/MS as previously described. Reference standard of the released drug was also analyzed at low, mid and high QC ranges, (1.5, 800, and 1500 ng/mL of conjugated drug respectively), via LC-MS/MS. Percent recovery of the drug released from the ADC at the LQC, MQC, and HQC ranges was evaluated by comparing the equivalent LC-MS/MS response of the recovered drug after protease digestion to the equivalent level of the unconjugated drug quantified at the LQC, MQC, and HQC ranges. FIG. 1B shows the percent drug recovery at the LQC, MQC, and HQC levels after papain and β-glucuronidase digestion. Drug recovery levels of around 100% were seen at each quality control level, indicating high recovery of the drug cleaved from the ADC. Therefore, the double digestion method resulted in both good enzymatic cleavage of the antibody to expose the glucuronide link and near complete cleavage of the drug from the ADC.

### Example 3

### Animal Sample Materials and Methods

### Animal Dosing and Sample Collection

In Example 4, cynomolgus monkeys were dosed intravenously (IV) with a pegylated CD228-targeting ADC containing a glucuronide linker on day 1 (hr 0). Animals were divided into 3 groups, group 1 was dosed at 0.1 mg/kg, group 2 was dosed at 0.3 mg/kg, and group 3 was dosed at 1.0 mg/kg. Blood samples were collected at 0.083 hrs, 0.5 hrs, 2 hrs, 6 hrs, 12 hrs, 24 hrs, 48 hrs, 96 hrs, 168 hrs, 336 hrs, 504 hrs and 672 hrs post dosing, stored on ice, and transferred to K₂EDTA tubes within 30 minutes of collection. Processed samples were stored at −80° C.

In Example 5, cynomolgus monkeys were dosed IV with 0.3 mg/kg, 1 mg/kg, or 3 mg/kg of a pegylated CD48-targeting ADC containing a glucuronide linker on day 1 (hrs 0). Whole blood samples were collected at 0 hr, 0.167 hrs, 2 hrs, 8 hrs, 24 hrs, 48 hrs, 96 hrs, 168 hrs, 240 hrs, 336 hrs, and 504 hrs post dosing, and processed to K₂EDTA plasma within 30 minutes of collection. Samples were stored at −80° C.

### Calibration and Quality Control Sample Preparation

Calibration standards and quality control standards were prepared on ice in like for like matrix as compared to the samples analyzed (i.e. calibration standards were prepared in cynomolgus plasma for analysis of cynomolgus monkey plasma samples) by spiking ADC reference standards (containing glucuronide linker) into control plasma samples. Serial dilution of the samples was performed to cover the validated calibration range of 4-1000 ng/mL. Internal standard (IS) working stocks of a deuterated ADC (d8-ADC) with an identical glucuronide linker chemistry were prepared by diluting the IS into loading buffer (TBS/0.1% Tween-20 with 0.1% BSA (w/v)). Drug loading quality control samples were also prepared by diluting the drug into the loading buffer.

### Protein A Binding

Protein A magnetic beads were mixed gently to ensure uniform suspension in storage solution, washed 3 times using loading buffer, and pelleted via an external magnet. After the third wash, beads were resuspended in loading buffer.

Unknown samples were thawed on ice, mixed, and 25 μL was transferred to an individual well of a 96-deep well LoBind plate. 25 μL of the internal standard working stock was added followed by an additional 200 μL of loading buffer to each well. The samples were mixed, and plates centrifuged settle sample at bottom of wells. 50 μL of solution from each well was transferred to an individual well of a new deep well LoBind plate containing 100 μL of loading buffer. 50 μL of previously washed and resuspended Protein A beads was added to each well. Plates were sealed and vortexed for at least 1 hour at room temperature to ensure binding of the IgG to beads. Each sample was washed three times with 1× TBS using a KingFisher Purification System (Thermo Fisher Scientific).

### Enzyme Digestion

For the first digestion step, beads with bound sample were transferred to a 96 deep well LoBind plate containing 200 μL Papain digestion solution (2.0 mg/mL papain, 0.242 mg/mL L-cysteine in 20 mM ammonium acetate) which was previously activated at 37° C. for 15 minutes. Plates were incubated for 1 hour at 60° C. with shaking.

For the second digestion step, 50 μL of the β-glucuronidase digestion solution (20,000 units/mL β-glucuronidase in 200 mM ammonium acetate, pH 4.5) was added to each well and plates were incubated overnight at 37° C. with mixing.

Once the digestions were complete, 50 μL of ethanol was added to each well. Samples were vortexed and transferred to a filterplate on top of a 96 deep well LoBind plate. Plates were centrifuged and 25 μL of 5% NH₄OH in methanol was added.

### Sample Analysis

Samples were analyzed for drug and d8-drug using LC-MS/MS with reversed phase chromatography, H2O+0.1% Formic acid and acetonitrile+0.1% Formic acid mobile phases, and a Sciex 6500 triple quadrupole mass spectrometer (SCIEX).

### Example 4

### CD228-Targeting ADC Exposure in Cynomolgus Monkey Samples

Cynomolgus monkeys were dosed with a CD228-targeting ADC for a pharmacokinetic 28 day time course study. Plasma samples collected from the animals were analyzed using the double digestion method described in Example 3 to determine the concentration and exposure of circulating CD228-targeting ADC in the animals.

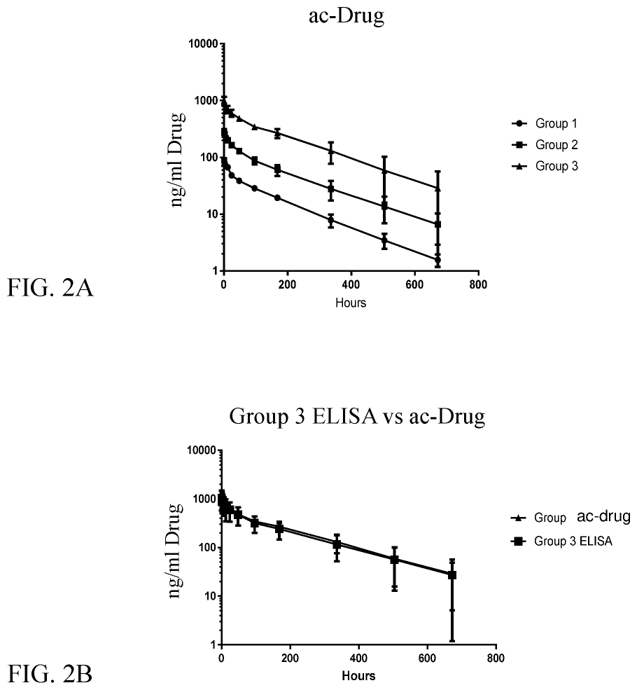

In each group, the antibody-drug concentrations decreased by about 1.5 logs over 28 days (FIG. 2A).

Next, the robustness of the assay developed in Example 3 was assessed. Plasma samples from Group 3 were tested with a genetic TAb ELISA (converted to ac-drug with a drug-to-antibody ratio (DAR) of 8), and the results compared to Group 3 drug levels from FIG. 2A. FIG. 2B shows the comparison results. Comparison of the ac-drug and ELISA analysis methods show that both methods resulted in comparable quantification of the circulating antibody conjugated drug. The results demonstrate a stably conjugated ADC and the robustness of both analytical methods.

### Example 5

### CD48-Targeting ADC Exposure in Cynomolgus Monkeys

Plasma samples collected in a cynomolgus monkey toxicology 15 day time course study were analyzed using the method described in Example 3 to determine the concentration and exposure of antibody conjugated drug in circulation in the animals. Three concentrations of CD48-targeting ADC were tested, 0.3 mg/kg, 1 mg/kg, and 3 mg/kg.

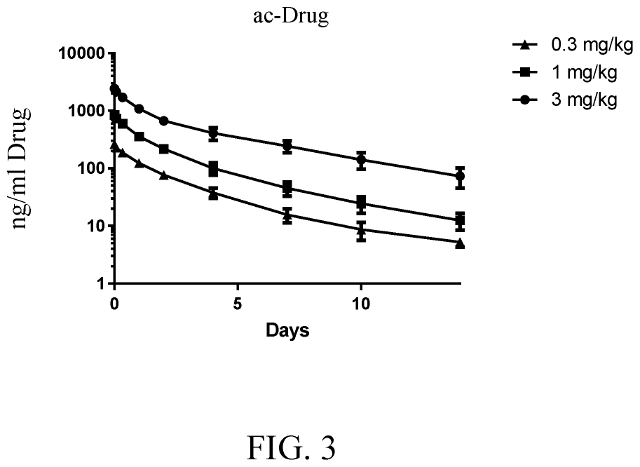

In each condition, the antibody-drug concentrations decreased by about 1 log over 15 days (FIG. 3), demonstrating a stably conjugated CD48-targeting ADC and the robustness of the analytical method.

### Example 6

### CD48-Targeting ADC Exposure in Humans

### Calibration and Quality Control Sample Preparation

In Example 6, cancer patients (humans) were dosed IV with a pegylated CD48-targeting ADC containing a glucuronide linker. Doses ranged from 0.15 to 0.6 mg/kg every 3 weeks. Blood samples were collected in K₂EDTA tubes, stored on ice, and processed to plasma within 30 minutes of collection. Processed samples were stored at −80° C.

Calibration standards and quality control standards were prepared on ice in like-for-like matrix as compared to the samples analyzed (i.e. calibration standards were prepared in human K₂EDTA plasma for analysis of human plasma samples) by spiking ADC reference standards (containing glucuronide linker) into control plasma samples. If needed, serial dilution of the samples into the validated calibration range of 0.5-1000 ng/mL was performed. Internal standard (IS) working stocks of a deuterated ADC (d8-ADC) with an identical glucuronide linker chemistry were prepared by diluting the IS into loading buffer (TBS/0.1% Tween-20 with 0.1% BSA (w/v)). Quality control samples were also prepared by diluting the drug into plasma.

### Protein A Binding

Protein A magnetic beads were mixed gently to ensure uniform suspension in storage solution, washed 3 times using loading buffer, and pelleted via an external magnet. After the third wash, beads were resuspended in loading buffer.

Unknown samples were thawed on ice, mixed, and 25 μL was transferred to an individual well of a 96-deep well LoBind plate. 25 μL of the internal standard working stock was added followed by an additional 200 μL of loading buffer to each well. The samples were mixed, and plates were centrifuged to settle sample at the bottom of wells. 50 μL of solution from each well was transferred to an individual well of a new deep well LoBind plate containing 100 μL of loading buffer. 50 μL of previously washed and resuspended Protein A beads was added to each well. Plates were sealed and vortexed for at least 1 hour at room temperature to ensure binding of the IgG to beads. Each sample was washed three times with 1× TBS using a KingFisher Purification System (Thermo Fisher Scientific).

### Enzyme Digestion

For the first digestion step, beads with bound sample were transferred to a 96 deep well LoBind plate containing 200 μL Papain digestion solution (2.0 mg/mL papain, 0.242 mg/mL L-cysteine in 20 mM ammonium acetate) which was previously activated at 37° C. for 15 minutes. Plates were incubated for 1 hour at 60° C. with shaking.

For the second digestion step, 50 μL of the β-glucuronidase digestion solution (20,000 units/mL β-glucuronidase in 200 mM ammonium acetate, pH 4.5) was added to each well and plates were incubated overnight at 37° C. with mixing.

Once the digestions were complete, 50 μL of ethanol was added to each well. Samples were vortexed and transferred to a filterplate on top of a 96 deep well LoBind plate. Plates were centrifuged and 25 μL of 5% NH₄OH in methanol was added.

### Sample Analysis

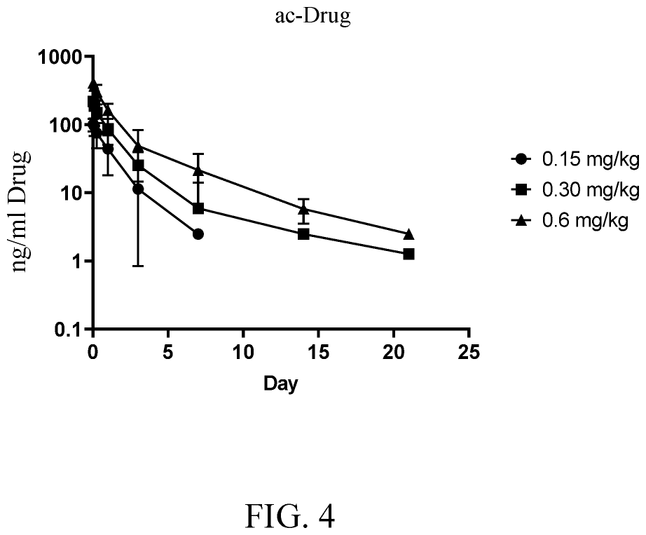

Samples were analyzed for drug and d8-drug using LC-MS/MS with reverse phase chromatography, H₂O+0.1% Formic acid and acetonitrile+0.1% Formic acid mobile phases, and a Sciex 6500 triple quadrupole mass spectrometer (SCIEX). As shown in FIG. 4, three concentrations of CD48-targeting ADC were tested, 0.15 mg/kg, 0.30 mg/kg, and 0.6 mg/kg. In each condition, the antibody-drug concentration gradually decreased demonstrating a stably conjugated CD48 targeting ADC and the robustness of the analytical method.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### Equivalents

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method for releasing an analytic target from a ligand-drug conjugate (LDC) in a sample, comprising:
providing a sample comprising an LDC, wherein said LDC comprises a ligand and an analytic target, wherein said analytic target comprises a drug molecule or a portion thereof, and wherein said analytic target is attached to said ligand by a linker comprising a glucuronide;
contacting said sample with a proteolytic enzyme under conditions that promote exposure of said glucuronide; and
contacting said sample with a glucuronidase, thereby inducing release of said analytic target from said LDC.

2. The method of claim 1, further comprising the steps of:
measuring an amount of said analytic target released from said LDC; and
determining a concentration of said drug molecule or said portion thereof in said sample using said amount of said released analytic target.

3. The method of claim 2, wherein the step of measuring said amount of said analytic target released from said LDC comprises subjecting said analytic target to liquid chromatography-mass spectrometry (LC-MS).

4. The method of claim 2, wherein the step of measuring said amount of said analytic target released from said LDC comprises subjecting said analytic target to liquid chromatography tandem mass spectrometry (LC-MS/MS).

5. The method of claim 3 or 4, wherein said liquid chromatography is high performance liquid chromatography (HPLC).

6. The method of any of claims 2-5, further comprising:
measuring said amount of said ligand in said sample; and
determining said concentration of said drug molecule or said portion thereof in said sample by using said measured amount of said ligand.

7. The method of any of claims 1-6, wherein said sample is obtained from a cell or a mammalian subject.

8. The method of claim 7, wherein the subject is a human subject.

9. The method of claim 7, wherein the sample is processed using a step selected from the group consisting of: affinity chromatography, size exclusion chromatography, ammonium sulfate precipitation, ion exchange chromatography, immobilized metal chelate chromatography, and immunoprecipitation.

10. The method of any of the above claims, wherein said ligand is an antibody or a functional fragment thereof and said LDC or said internal standard are collected by affinity chromatography, size exclusion chromatography, ammonium sulfate precipitation, ion exchange chromatography, immobilized metal chelate chromatography, or immunoprecipitation.

11. The method of claim 10, wherein said ligand is an antibody or a functional fragment thereof and said LDC or said internal standard are collected by contacting said sample with an antigen, an antibody idiotype, an affinity magnetic bead, or a resin.

12. The method of claim 11, wherein said resin is selected from a Protein A resin, a Protein G resin, and a Protein L resin.

13. The method of any of claims 1-12, further comprising:
adding to said sample a fixed amount of an internal standard, where said internal standard comprises said ligand and a second analytic target, wherein said second analytic target is a labeled derivative of said LDC;
contacting said sample with said proteolytic enzyme under conditions that promote exposure of said glucuronide;
contacting said sample with said glucuronidase, thereby inducing release of said analytic target from said LDC and said second analytic target from said internal standard;
measuring an amount of said second analytic target released from said internal standard; and
measuring said amount of said analytic target released from said LDC based on said amount of said second analytic target released from said internal standard.

14. The method of claim 13, wherein said amount of said analytic target released from said LDC is determined by using said amount of said second analytic target released from said internal standard, wherein said amount of said analytic target released from said LDC correlates with a concentration of said drug molecule conjugated to an antibody in said LDC in said sample.

15. The method of any of claims 2-14, wherein the step of measuring said amount of said analytic target released from said LDC comprises using a standard curve generated from said LDC.

16. The method of any of claims 13-15, wherein said second analytic target has a different molecular weight than said analytic target.

17. The method of any of claims 13-15, wherein said second analytic target has a different isobar than said analytic target.

18. The method of any of claims 13-17, wherein said internal standard comprises a version of said LDC further comprising an isotopic label.

19. The method of claim 18, wherein said isotopic label is stable or non-stable.

20. The method of claim 19, wherein said isotopic label is deuterium or carbon 13.

21. The method of any of claims 1-20, wherein said glucuronidase comprises β-glucuronidase.

22. The method of any of claims 1-21, wherein said first or second sample is contacted with β-glucuronidase at a concentration of at least 10,000 units/mL.

23. The method of claim 22, wherein said first or second sample is contacted with β-glucuronidase at a concentration of no more than 100,000 units/mL.

24. The method of any of claims 1-23, wherein said contacting said first or second sample with said glucuronidase occurs at a pH of about 5.

25. The method of any of claims 1-24, wherein said drug molecule is monomethyl auristatin E (MMAE).

26. The method of any of claims 1-25, wherein said proteolytic enzyme is papain or trypsin.

27. The method of claim 26, wherein said proteolytic enzyme is papain.

28. The method of claim 26, wherein said proteolytic enzyme is trypsin.

29. The method of any of claims 1-28, wherein said contacting said first or second sample with said glucuronidase is at least six (6) hours at 37° C.

30. A method of determining stability of the ligand-drug conjugate (LDC), comprising:
obtaining a first sample and a second sample from a single source at different time points;
analyzing said LDC in said first sample and said second sample by the method of any of claims 2-29, thereby determining the amount of said analytic target released from said LDC in said first sample and said second sample; and
determining stability of said LDC by comparing the amount of said released analytic target in said first sample and said second sample.

31. The method of claim 30, wherein said comparing comprises determining a ratio of said amount of said released analytic target and said ligand in said first and said second samples.

32. The method of any of claims 1-31, wherein said first sample has a volume of at least 20 μL.

33. The method of any of claims 1-32, wherein said sample, said first sample, or said second sample is a biological sample derived from mammalian tissue or aqueous mammalian fluid.

34. The method of claim 33, wherein said biological sample is obtained from one of the following: plasma, serum, blood, tissue, tissue biopsy, feces, urine.

35. The method of claim 33 or 34, wherein said biological sample is obtained from plasma.

36. The method of claim 35, wherein said plasma was treated with said LDC.

37. The method of any of claims 35-36, wherein said plasma is from a mammalian subject to whom said LDC has been administered.

38. The method of claim 37, wherein the subject is a human subject.

39. The method of claim 37, wherein the subject is a rat or cynomolgus monkey subject.

40. The method of any of claims 1-39, wherein said ligand is an antibody comprising an Fc region.

41. The method of claim 14 or 40, wherein said antibody is an anti-CD48 antibody or an anti-CD228 antibody.

42. A method for quantifying a ligand-drug conjugate (LDC) in a sample, comprising:
providing a sample comprising an LDC, wherein said LDC comprises a ligand and an analytic target, wherein said analytic target comprises a drug molecule or a portion thereof, wherein said analytic target is attached to said ligand by a linker comprising a glucuronide;
adding to said sample an internal standard, wherein said internal standard is a labeled derivative of said LDC and comprises a second analytic target;
contacting said LDC and said internal standard with a proteolytic enzyme under conditions that promote exposure of said glucuronide in said LDC;
contacting said LDC and said internal standard with a glucuronidase, wherein said glucuronidase induces release of said analytic target from said LDC and said second analytic target from said internal standard; and
determining an amount of said analytic target released from said LDC and an amount of said second analytic target released from said internal standard, wherein said amount of said analytic target released from said LDC correlates with said amount of said LDC in said sample.

43. The method of claim 42, further comprising extracting said LDC and said internal standard from said sample prior to contacting said LDC and said internal standard with said proteolytic enzyme.

44. The method of any of claims 42-43, wherein the step of determining said amount of said analytic target released from said LDC and said amount of said second analytic target released from said internal standard comprises subjecting said analytic target and said second analytic target to liquid chromatography-mass spectrometry (LC-MS).

45. The method of any of claims 42-43, wherein the step of determining said amount of said analytic target released from said LDC and said amount of said second analytic target released from said internal standard comprises subjecting said analytic target and said second analytic target to liquid chromatography tandem mass spectrometry (LC-MS/MS).

46. The method of claim 44 or 45, wherein the liquid chromatography is high performance liquid chromatography (HPLC).

47. The method of any of claims 42-46, further comprising:
measuring an amount of said ligand in said first or second sample; and
determining a concentration of said drug molecule or said portion thereof in said first or second sample by using said measured amount of said ligand.

48. The method of any of claims 43-41, wherein the step of extracting said LDC and said internal standard is performed by at least one of: affinity chromatography, size exclusion chromatography, ammonium sulfate precipitation, ion exchange chromatography, immobilized metal chelate chromatography, and immunoprecipitation.

49. The method of any of claims 42-48, wherein said ligand is an antibody or a functional fragment thereof and said LDC or said internal standard are extracted by contacting said sample with a resin selected from a Protein A resin, a Protein G resin, and a Protein L resin.

50. The method of any of claims 42-49, wherein said amount of said analytic target released from said LDC is determined by using said amount of said second analytic target released from said internal standard, wherein said amount of said analytic target released from said LDC correlates with a concentration of said drug molecule conjugated to an antibody in said LDC in said sample.

51. The method of any of claims 42-50, wherein said amount of said analytic target released from said LDC is determined by using a standard curve generated from said LDC.

52. The method of any of claims 42-51, wherein said second analytic target has a different molecular weight than said analytic target.

53. The method of any of claims 42-52, wherein said internal standard comprises a version of said LDC further comprising an isotopic label.

54. The method of claim 53, wherein said isotopic label is stable or non-stable.

55. The method of any of claims 53-54, wherein said isotopic label is deuterium or carbon 13.

56. The method of any of claims 42-55, wherein said glucuronidase comprises β-glucuronidase.

57. The method of any of claims 42-56, wherein said sample is contacted with β-glucuronidase at a concentration of at least 10,000 units/mL.

58. The method of claim 57, wherein said sample is contacted with β-glucuronidase at a concentration of no more than 100,000 units/mL.

59. The method of any of claims 42-58, wherein said contacting said sample with said glucuronidase occurs at a pH of about 5.

60. The method of any of claims 42-59, wherein said drug molecule is monomethyl auristatin E (MMAE).

61. The method of any of claims 42-60, wherein said proteolytic enzyme is papain or trypsin.

62. The method of any of claims 42-61, wherein said proteolytic enzyme is papain.

63. The method of any of claims 42-61, wherein said proteolytic enzyme is trypsin.

64. The method of any of claims 42-63, wherein said contacting said sample with said glucuronidase is at least six (6) hours at 37° C.

65. The method of any of claims 1-64, wherein said sample has a volume of at least 20 μL

66. The method of any of claims 42-64, wherein said sample is a biological sample derived from mammalian tissue or aqueous mammalian fluid.

67. The method of claim 66, wherein said biological sample is obtained from one of the following: plasma, serum, blood, tissue, tissue biopsy, feces, and urine.

68. The method of any of claims 66-67, wherein said biological sample is obtained from plasma.

69. The method of claim 68, wherein said plasma was treated with said LDC.

70. The method of any of claims 68-69, wherein said plasma is from a human subject to whom said LDC has been administered.

71. The method of any one of claims 1-70, wherein said ligand is an antibody comprising an Fc region.

72. The method of any of claim 49 or 71, wherein said antibody is an anti-CD48 antibody or an anti-CD228 antibody.
